(19) United States
(12) Patent Application Publication
(10) Pub. No.: US 2006/0034887 A1
(43) Pub. Date: Feb. 16, 2006

(54) Implantable structure for prolonged and controlled release of an active principle

(76) Inventor: Edouard Pelissier, Devecey (FR)

Correspondence Address: LUCAS & MERCANTI, LLP, 475 PARK AVENUE SOUTH, 15TH FLOOR, NEW YORK, NY, 10016, US

(21) Appl. No.: 10/532,535

(22) PCT Filed: Oct. 29, 2003
(86) PCT No.: PCT/FR03/03221

§ 371 Date: May 16, 2005

(30) Foreign Application Priority Data
Oct. 30, 2002 (FR) 0213623

(51) Int. Cl. A61F 2/00

(52) U.S. Cl. 424/426

(57) ABSTRACT

The invention relates to an implantable structure of flexible consistency for the sustained and controlled release of an active substance, consisting of a bioresorbable support and an active substance, in which the bioresorbable support is formed of a material comprising an aliphatic polyester of therapeutic value as the main component. The invention further relates to a process for the manufacture of this structure.

The present invention relates to a bioresorbable implantable structure for the controlled release of an active substance in an intracorporeal lesion, especially a surgical incision, and to a process for the manufacture of such a structure.

The invention is applied in the field of medicine and surgery.

The treatment of pain, especially in a hospital environment and particularly after a surgical intervention, is nowadays increasingly a major concern of medical staff.

Postoperative pain is due to the direct stimulation, by the surgical trauma, of the free nerve endings present in all tissues, and to the release, by the traumatized tissues, of algesiogenic substances which sustain the pain by direct stimulation of the nerve endings and by lowering the activation threshold of the nociceptive receptors.

The treatment of postoperative pain with analgesics is only a symptomatic treatment which attenuates the perception of pain at the nerve centers without acting at the source.

Local anesthetics have a direct action on the nerve endings, totally interrupting the transmission of pain by the nerve fibers. Their efficacy is such that it is possible to incise and operate on a zone infiltrated by local anesthesia.

It has been demonstrated that infiltration of the wound by a local anesthetic after an intervention effectively suppresses the postoperative pain, but the effect wears off after a few hours when the product is absorbed (1-5).

Other studies (6-10) have confirmed the value of the method of continuous irrigation of the wound with a local anesthetic by means of a catheter.

However, although effective, this method has the disadvantage of involving bulky equipment which restricts activity and hence nullifies the advantage of pain reduction.

U.S. Pat. No. 6,063,405 relates to the preparation of a delivery system for the sustained release of an active substance such as an anesthetic, said system being formed of a polymer matrix suspended or dissolved in water and being intended for injection.

Furthermore, patent application WO 00 50004 discloses a composition in the form of a gel or a solution of low viscosity at room temperature for the administration of a local anesthetic in a surgical operation, especially in urological surgery.

However, this system and this composition have only a limited effect over time.

The object of the invention is to overcome these disadvantages and provide a structure for the gradual and controlled release of an active substance at an intracorporeal incision.

Such a structure must be able to be positioned rapidly and easily at the time of repair, for example after a surgical incision. It must also be resorbed as rapidly as possible.

Thus, according to a first aspect, the invention relates to an implantable structure of flexible consistency for the sustained and controlled release of an active substance, said structure consisting of a bioresorbable support and an active substance, said active substance being intimately associated with the support.

The basis of the present invention is therefore the fact that the implantable structure possesses a flexible consistency and exhibits a cohesion (interlocking) between the active substance and the bioresorbable support material that is induced by the wettability of one of the components of the structure.

According to the invention, the main component of the bioresorbable support is an aliphatic polyester of therapeutic value, i.e. which is biocompatible, bioresorbable and well tolerated and does not cause adverse effects such as local irritation, allergic reaction, immunological reaction or systemic toxicity. Examples of such polyesters which may be mentioned are poly(a-hydroxy acids) derived from lactic acid (LA) and/or glycolic acid (GA), particularly the lactic acid/glycolic acid copolymers of the formula PLAₓGA_{y} (x and y varying in the range 0 to 100 and specifying the percentages of lactic and glycolic acid units, respectively). This notation can also specify the diastereoisomeric form of the lactic acid unit (D, L, DL).

Materials of amorphous structure and of low molecular weight will generally be preferred.

It is advantageous to use a DL PLA-GA copolymer in which the weight ratio between the lactic and glycolic acid units ranges from about 80/20 to 20/80, preferably 70/30 to 30/70. A copolymer comprising equal proportions of lactic and glycolic acids is very particularly preferred.

In the family of aliphatic polyesters of therapeutic value, there may also be mentioned poly-ε-caprolactone (PCL), which has a semicrystalline structure (degree of crystallinity of around 56%), polyorthoesters, poly-p-dioxanones (DS) or polytrimethylene carbonate (TMC). Mixtures of these various polymers in all proportions, such as a PCL/PLAₓGA_{y} mixture, can also be envisaged.

The implantable structure according to the invention has a good flexibility. In terms of the present invention, “flexibility” is understood as meaning that the Tg (glass transition temperature) of the support material is below or equal to about 15° C., so said material can easily be manipulated without risk of breaking when it is positioned in the operative wound.

This flexibility is a characteristic of polymer materials with so-called viscoelastic properties. For one and the same material heated to temperatures T, this flexibility represents the transition, as a function of T, between so-called “rigid-elastic” structures at temperatures below the glass transition temperature (T<Tg) and so-called “elastic-rubbery” structures at temperatures above the glass transition temperature (T>Tg). This property is commonly studied with the aid of viscoelastimeters, in which the aforesaid transition is defined by the characteristic variation with temperature of an elastic modulus of the Young's modulus or shear modulus type.

If the aliphatic polyester of therapeutic value is a lactic acid/glycolic acid copolymer whose Tg is close to room temperature, the flexibility of the support material is adjusted, in a manner well known to those skilled in the art, by the addition of a biocompatible plasticizer. Examples which may be mentioned in particular are lactic acid, lactic acid oligomers, commonly denoted by OLA, and mixtures of these compounds. Any biotolerated products from the family of alcohols, polyethylene oxides, polyethylene glycols and citrates which have a solubility parameter similar to that of the polymer of the support of the implantable structure may also be mentioned. The plasticizer is generally added in an amount ranging from about 0.5% to 20% by weight, preferably from 5 to 15% by weight, based on the weight of the support.

If the aliphatic polyester of therapeutic value is poly(ε-caprolactone), the kinetics of release and degradation of the implantable structure have to be increased by adding water-soluble materials.

The additives can be those of low molecular weight, such as salts (sodium chloride, sodium phosphate) or sugars (sucrose, lactose), or those selected from the large family of surfactants (e.g. sodium laurylsulfonate or laurylsulfate). Other, preferred possibilities are hydrophilic polymers (polyethylene oxide (PEO), polyethylene glycol (PEG), polyvinyl alcohol (PVA)), polysaccharides, such as those marketed under the name DEXTRAN® (α-1,6-glucan) or PLURONIC®, or cellulose derivatives (methyl cellulose, hydroxypropyl cellulose).

The amount of water-soluble material added must remain limited to about 20% by weight and must preferably range from about 2 to 10% by weight, based on the weight of the support.

The active substance which can be used within the framework of the invention is not restricted to one particular active substance and can advantageously be selected from local anesthetics, morphine or non-morphine analgesics, healing factors, anti-inflammatories, antibiotics, antifungals, corticoids, hormones, antimitotics and growth factors. A mixture of active substances is also possible for the purpose of:
modulating the efficacy of the active substances over time,
ensuring different cumulative therapeutic functions.

Thus it is possible to conceive of applying anti-inflammatories or corticoids to or in the vicinity of a joint, antiseptic and/or healing substances to a chronic wound, antimitotics to an inextirpable tumor or metastases, or stimulating substances to a nerve structure.

Particularly preferably, the active substance is a local anesthetic such as lidocaine, bupivacaine, benzoin, tetracaine, mepivacaine or ropivacaine. A substance such as clonidine or fentanyl can be added to the local anesthetic in order to prolong its action.

The amount of active substance(s) does not generally exceed 60% of the weight of the support and varies according to the precise nature of the active substance and the intended therapeutic objective.

The biodegradable structure according to the invention can be implanted in suture planes of incised tissues and affords an in situ delivery of active substance at a defined concentration for a period of time specific to the intended therapeutic objective.

This structure withstands sterilization and storage under different climatic conditions.

In the case where the active substance is a local anesthetic, the implantable structure according to the invention makes it possible to eliminate or minimize pain, and very particularly postoperative pain, with all the inherent advantages: ideal comfort for the patient, minimization of the postoperative care, reduction of the risk of thromboembolism by the early resumption of walking, shortening of the period of hospitalization and hence reduction of the costs. It also promotes the practice of outpatient surgery and a rapid resumption of activity by the patient.

The implantable structure according to the invention is manufactured by means of a thermomechanical shaping process which produces a cohesion (interlocking) between the active substance and the bioresorbable support material that is induced by the wettability of one of the components of the structure.

If the active substance is passed through a liquid phase, which induces this wettability, when the temperature rises above its melting point imposed by one of the steps of the manufacturing process, the desired cohesion is implicit.

If the active substance remains in its solid form throughout the manufacturing process, it is the support material which has to be passed through a liquid or viscous phase during the manufacturing process.

If the melting point of the active substance and the glass transition temperature or melting point of the support material are similar, it is possible for both the active substance and the support material to undergo this phase change concomitantly.

Said phase change to obtain the desired interlocking is generally effected in a so-called “transfer chamber”.

Thus, according to a second aspect, the invention relates to a process for the manufacture of the implantable structure described above, said process comprising the following steps:

a) homogeneous mixing of the component products of the structure,

b) passage of some or all of the resulting mixture through the liquid and/or viscous state, with or without applied pressure, in a transfer chamber, and

c) shaping of the implantable structure under pressure from this intermediate state.

These different steps can be complemented, if necessary, with a final heat treatment.

In step a), the products, initially in the solid state, are advantageously subjected beforehand to a desiccation treatment.

The phase change is effected in step b). This phase change is of one of the following types:
solid-liquid in the case where it is induced preferentially by the active substance alone or the support material alone,
solid-viscous or solid-viscous-liquid in the case where it is induced preferentially by an amorphous or, respectively, semicrystalline aliphatic polyester material, or
liquid-liquid in the case where it is induced by the concomitant melting of the active substance and the support material.

When the process has ended, the shaped products are released from the mold, preferably onto a cooled plate.

It is therefore seen that the process of the invention does not involve a solvent in the different steps indicated above.

If there is a large difference between the melting point (Tm) of the active substance and the glass transition temperature or melting point of the aliphatic polyester (according to whether it is amorphous or semicrystalline), the phase change of step b) is advantageously effected at a temperature between the Tm of the active substance and the Tg or Tm of the aliphatic polyester, and preferably at a temperature close to the Tm of the active substance.

If there is a small difference between the Tm of the active substance and the Tg or Tm of the aliphatic polyester (approximately in the order of at most 10 to 15° C.), the phase change of step b) is advantageously effected at a temperature that is above both the Tm of the active substance and the Tm or Tg of the aliphatic polyester.

The process according to the invention affords a homogeneous composite structure with coherent interfaces (i.e. without interfacial loss of cohesion) which additionally has a low melting point or glass transition temperature (below that of the aliphatic polyester).

Examples which may be mentioned of the process according to the invention are those described especially in “La mise en forme des matières plastiques (The shaping of plastics): J. F. AGASSANT, P. AVENAS, J. Ph. SERGENT, publ. Lavoisier 1989” or in “Matières plastiques (Plastics): J. P. TROTIGNON, J. VERDU, A. DOBRACZYNSKI, M. PIPERAUD, publ. Nathan 1996”, particularly the processes of the following types:
compression-transfer molding,
injection-transfer molding,
extrusion or spinning with a preliminary transfer step.

It is preferable to use the process of the compression-transfer molding type, which consists in introducing a material, in a given state of fluidity, into the cavity of a mold under pressure, said process conventionally breaking down into four steps:
plastification: the material, introduced into a crucible first, is partially or totally heated to a homogeneous fluid state (phase change),
injection: this fluidized material is introduced into the mold by means of a piston,
shaping: the material can then be shaped in the mold with rapid curing kinetics,
final release from the mold.

Advantageously, the particle size of the mixture of starting materials is controlled within the range between about 5 and 150 μm, preferably between about 10 and 50 μm. As a general rule, it is advisable to use mechanical mills or air-jet mills. It is further recommended to work in shaping temperature ranges in which the active substance retains its structural integrity and its therapeutic properties.

Injection-transfer molding is based on the same experimental approach. Extrusion-transfer, incidentally, is a known technology.

The morphology of the implantable structures can be varied and result in the manufacture of yarns, films, hanks, ribbons (especially of parallelepipedal shape with a square or rectangular base), slivers, woven or non-woven fabrics, plates, catheters, tablets or even sheets. It is possible to imagine other shapes, for example a film which could be applied in breast surgery or anal surgery as well as in the treatment of burns and other skin lesions. A structure in which the active substance(s) were incorporated in a suture thread could also be envisaged.

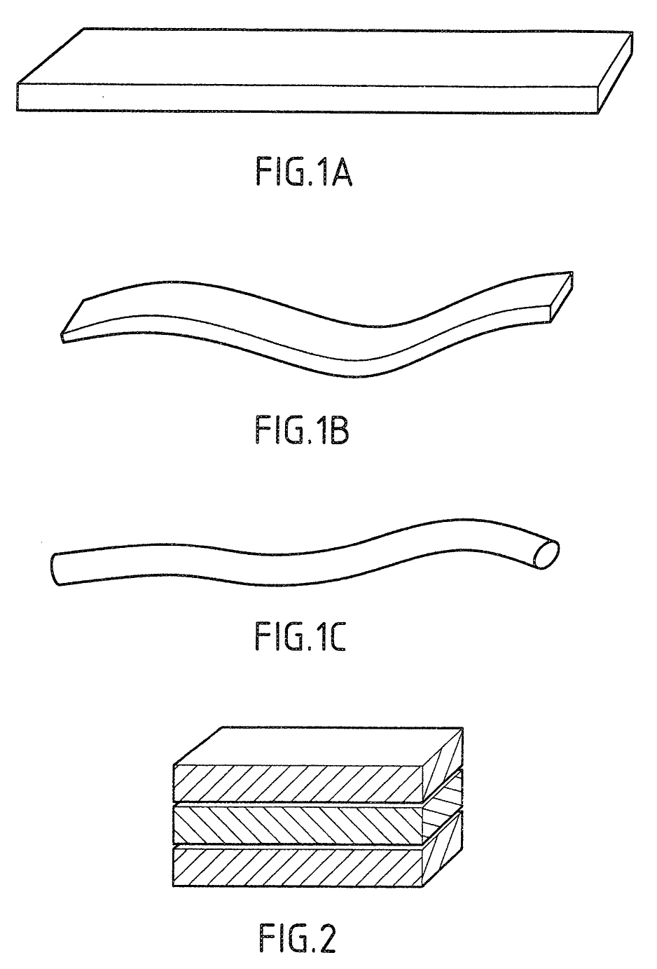

Various structures according to the invention are shown in FIGS. 1A (ribbon), FIG. 1B (crimped ribbon) and FIG. 1C (hank).

Furthermore, mixed composite structures comprising the implantable structure according to the invention can be produced in order to have several complementary release kinetics, for example rapid release kinetics in the first few hours after surgery, followed by retarded release kinetics.

The specific morphology resulting from this is conventionally effected via the creation of sandwich structures such as that shown in FIG. 2.

Advantageously, the exchange surface of the bioresorbable product with the interstitial liquid can be increased in order to accelerate its biodegradability and thereby favor the permeation to fluids and the active substance, for example by creating structures of defined geometric shape (cylinder, parallelepiped) with a surface topography having undulations or surface roughnesses formed of repeated or random geometric patterns, in order to increase the specific surface area of the implantable structures.

The insertion site for the implantable structure depends on the type of lesion or incision:

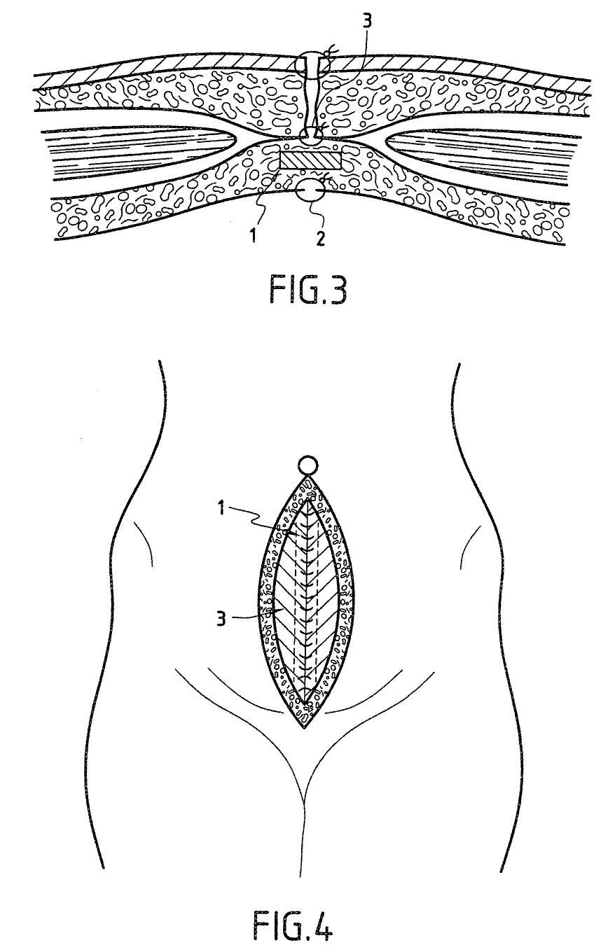

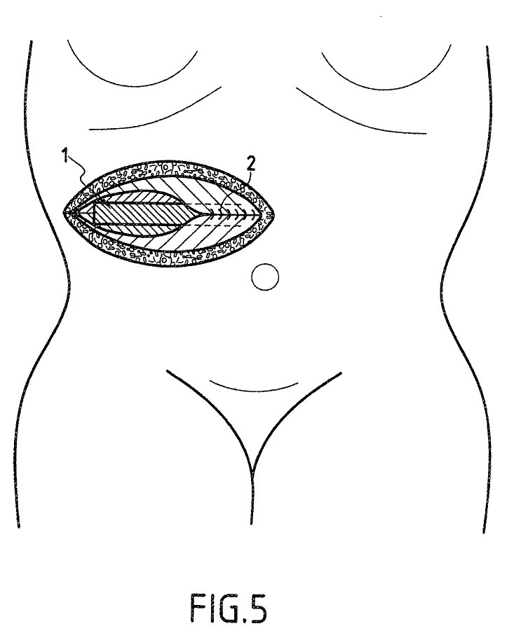

FIGS. 3 and 4 schematically show a median laparotomy: the implantable structure (1) is placed longitudinally, relative to the incision, between the suture plane of the peritoneum (2) and that of the aponeurosis (3), in the preperitoneal space.

FIG. 5 schematically shows a transverse laparotomy: the implantable structure (1) is placed in the muscle chamber, on the deep face of the aponeurotic suture plane (2).

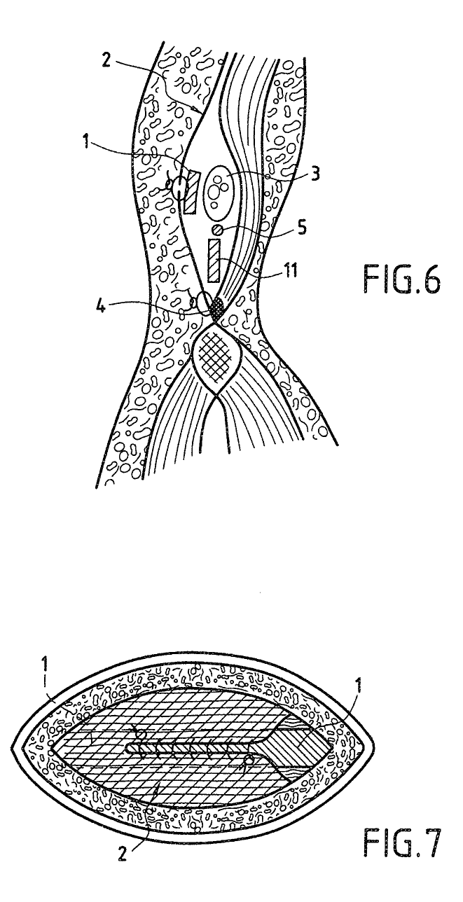

FIG. 6 schematically shows a herniorraphy: one piece of the implant (1) is placed on the deep face of the aponeurosis of the abdominal external oblique muscle (2) and another piece (11) is placed on the lower margin of the spermatic cord (3), in contact with the deep suture plane (4) and the genital branch of the genitofemoral nerve (5).

FIG. 7 is an anteroposterior section of the diagram of FIG. 6.

For other types of incisions, in the limbs or thorax, the implant will generally be placed in contact with muscular or aponeurotic sutures.

In the case of variceal strippings, the implant can be drawn by the stripper along the course of the stripping where it will be left in place.

Other shapes adapted to different types of surgery can subsequently be designed, especially in the form of a film for large detachments of cells.

Those skilled in the art will easily understand from the present description that the dimensions of the implantable structure and the amount of active substance to be incorporated into this structure will depend on the nature of the application envisaged.

The amount of active substance released depends on the weight of the bioresorbable support and on the initial concentration of this active substance in the support.

By way of indication, for the treatment of postoperative pain, the amount of local anesthetic of the lidocaine type is about 3 g for the continuous release of at most about 600 mg of said product per twenty-four hour period over five days.

This amount can be reduced for certain local anesthetics recognized as having a higher activity, such as bupivacaine, mepivacaine and ropivacaine.

By way of indication, the implants according to the invention generally have a length of between 3 and 25 cm and preferably of about 7 cm, this size being appropriate for many common incisions. If, for more major interventions requiring 15 to 20 cm incisions, it is desired to release the same daily dose of local anesthetic for the same period of time, and if the composition of the implant is the same, it is obviously necessary to reduce its other two dimensions:
For a 14 cm long and 0.22 cm thick implant of equal weight and volume, the width must be 0.75 cm. It is also possible to use two implants of 7×0.22×0.75 cm.
For a 21 cm long and 0.22 cm thick implant, the width must be 0.5 cm. It is also possible to use three implants of 7×0.22×0.5 cm.

It is also possible to release more active product by increasing the length of the implant, which offers the possibility of a larger volume for a thickness and width of the same order as in the basic example. In this case, the composition of the implant is quite obviously modified as a consequence.

The invention will be described in greater detail with the aid of the Examples below, which are given purely by way of illustration. l EXAMPLE 1

### Influence on the Tg of Adding a Plasticizer

Table 1 below collates the glass transition temperatures (Tg) and melting points (Tm) of two polymers that can be used as bioresorbable supports within the framework of the invention, namely PLA₅₀GA₅₀, an intrinsically biodegradable, amorphous material, and PCL, a semicrystalline material considered to be of low biodegradability.

The differences in Tg values recorded on the PLA₅₀GA₅₀ are linked to their nominal composition and in particular to their molecular weight (75,000 g/mol for the copolymer marketed by PURAC, 65,000 g/mol for the copolymer marketed by MEDISORB).

As a general rule, the Tg value is systematically evaluated for each batch of PLAGA.

TABLE 1

| Material | Tg (° C.) | Tm (° C.) |
|---|---|---|
| PLA₅₀GA₅₀ | 31 | |
| (MEDISORB) | | |
| PLA₅₀GA₅₀ | 45 | |
| (PURAC) | | |
| PCL | −60 | 60 |

Table 2 gives the Tg values (° C.) of the PLA₅₀GA₅₀ copolymers with 5, 10 or 15% by weight of added lactic acid.

TABLE 2

| % of lactic | PLA₅₀GA₅₀ | PLA₅₀GA₅₀ |
|---|---|---|
| acid | (MEDISORB) | (PURAC) |
| 0 | 31 | 45 |
| 5 | 20 | 41 |
| 10 | 15 | 20 |
| 15 | 3 | 16 |

The results in Tables 1 and 2 show on the one hand that PCL affords a support that is flexible at room temperature without a complementary addition, and on the other hand that it is possible to control the “flexibility” of the PLA₅₀GA₅₀ at room temperature by adding a plasticizer.

### EXAMPLE 2

### Optimization of the Shaping Conditions for Bioresorbable Structures

The object of this Example is to manufacture a ribbon of parallelepipedal shape that theoretically affords the controlled release of 500 mg/d of active substance for 3 days, i.e. 1500 mg in total.

A check was made beforehand to ensure that it was possible to incorporate about 50% of active substance in 50% of support material into the implantable structure while preserving the desired flexibility, irrespective of the chosen shaping process.

The total weight of the implant is 3 g under these conditions. For an estimated mean density of the implant of 1.3, the volume of the implant will be 2.31 cm³. This volume corresponds e.g. to a parallelepiped of the following dimensions:
imposed length: 7 cm
imposed width: 1.5 cm
thickness: 0.22 cm (2.2 mm)

As the particle size distribution of local anesthetics generally ranges from 10 to 500 μm, it is necessary first to grind these pulverulent products and then to pass them through an oven (e.g. one hour at 40° C.) to give a final particle size range of 10-50 μm.

A/ The following general operating conditions were chosen for the PLA₅₀GA₅₀ copolymer to give this particular morphology by compression-transfer molding:
plastification temperature: 80° C.-90° C.,
mold injection pressure: 60 bar-100 bar,
final cooling on Teflon-coated cooling plate before release from the mold.

B/ For poly(ε-caprolactone) the conditions for shaping by extrusion using a single-screw extruder marketed by SCAMIA are respectively 65, 80 and 120° C., this being for a stretching speed, expressed in meters per minute, that makes it possible to collect the extruded structures on a conveyor belt traveling at a speed of 1 m/min.

By way of indication, the viscosity of the PCL of molecular weight 37,000 g/mol was determined at these different temperatures (cf. Table 3) with the aid of an apparatus marketed by HAAKE under the name RHEOSTRESS RS 150. The tests describe the classical so-called NEWTON relationship, which relates the applied stress r (expressed in Pascal: Pa) to the shear rate \dot{γ}=dγ/dt (second⁻¹, s⁻¹): τ=η\dot{γ}. The viscosity η is expressed in Pascal seconds (Pa.s).

The experiments were conducted arbitrarily at an imposed \dot{γ} in the range 0.1 s⁻¹ to 200 s⁻¹ or at an imposed τ in the range 1 Pa to 12,000 Pa.

TABLE 3

| T (° C.) | τ_{imposed} | \dot{γ}_{imposed} |
|---|---|---|
| 65 | 1550 | 1600 |
| 80 | 980 | 980 |
| 120 | 360 | 350 |

Raising the temperature makes it possible to obtain a material of decreasing viscosity, i.e. of increasing fluidity, and harmoniously favors the association between the active substance and the support.

### EXAMPLE 3

### In Vitro Kinetics for the Release of Lidocaine from an Implant Based on PLA₅₀GA₅₀

These kinetics were measured on 3 g of ribbon prepared under the operating conditions of Example 2A/ and capable of releasing 1500 mg of lidocaine (manufactured by compression-transfer molding). This ribbon is immersed in 500 ml of PBS of pH 7.4 at a temperature of 37° C., with magnetic stirring, the immersion medium being changed every 24 h so that it does not become saturated with released product.

Compositions respectively containing the following percentages by weight:
85% of PLA₅₀GA₅₀ and 15% of lactic acid (85/15) or
90% of PLA₅₀GA₅₀ and 10% of lactic acid (90/10) or
95% of PLA₅₀GA₅₀ and 5% of lactic acid (95/5) were tested.

The values of the slopes of the release curves are collated in Table 4.

The in vitro method of determining the anesthetic released from the implantable structure is UV spectrometry (PERKIN ELMER Lambda 20). The characteristic line is situated at a wavelength of 263 nm. The determination is performed in continuous or discontinuous mode, the immersion bath being changed every 24 h.

TABLE 4

| PLA₅₀GA₅₀/lactic | | | | |
|---|---|---|---|---|
| acid composition | mg/24 h | mg/48 h | mg/72 h | mg/96 h |
| 85/15 | 600 | 900 | 1200 | 1500 |
| 90/10 | 500 | 900 | 1200 | 1500 |
| 95/5 | 450 | 800 | 1100 | 1400 |

If the implant is first subjected to a heat treatment at 40° C. for 2 hours, the behavior of the release kinetics per 24 h period is seen to be more homogeneous, being in the order of 350 mg/24 h.

### Example 4

### In vitro Kinetics for the Release of Lidocaine from an Implant Based on PCL Produced by Extrusion

A/ Poly(ε-caprolactone) (PCL), Molecular Weight=37,000 g/mol, with Different Percentages of Lidocaine

The tests correspond to the following mixtures:
test 1: 8 g of PCL+2 g of lidocaine
test 2: 6 g of PCL+4 g of lidocaine
test 3: 5 g of PCL+5 g of lidocaine

These tests are performed under the operating conditions of Example 2B/, only at a temperature of 80° C.

In the steady state, the values of the slopes of the “lidocaine concentration in g/l as a function of time” curves are shown in Table 5.

TABLE 5

| | |
|---|---|
| test 1 | 60 mg/24 h |
| test 2 | 254 mg/24 h |
| test 3 | 500 mg/5 h |

B/ PCL, Molecular Weight=37,000 g/mol, with a Constant Percentage of Lidocaine

The tests whose results are collated in Table 6 are performed under the operating conditions of Example 2B/ on implants composed of the same amount of PCL (8 g) and lidocaine (2 g) but extruded at different temperatures.

TABLE 6

| Extrusion temperature (° C.) | Slope |
|---|---|
| 65 | 60 mg/24 h |
| 80 | 320 mg/24 h |
| 100 | 320 mg/24 h |
| 120 | 360 mg/24 h |

Table 6 shows that the optimized association between the active substance and the support appears at 80° C. and above.

C/ PCL, Molecular Weight=37,000 g/mol, and PCL, Molecular Weight=10,000 g/mol, with a Constant Percentage of Lidocaine

A mixture of PCL of molecular weight 37,000 g/mol with an oligomer of the same material of molecular weight 10,000 g/mol was used. The tests correspond to the following initial mixtures:
test 1: 9 g of PCL (molecular weight 37,000 g/mol)+1 g of PCL (molecular weight 10,000 g/mol)+1 g of lidocaine
test 2: 8 g of PCL (molecular weight 37,000 g/mol)+2 g of PCL (molecular weight 10,000 g/mol)+1 g of lidocaine
test 3: 7 g of PCL (molecular weight 37,000 g/mol)+3 g of PCL (molecular weight 10,000 g/mol)+1 g of lidocaine

In the steady state, the values of the slopes of the release curves are shown in Table 7.

TABLE 7

| | |
|---|---|
| test 1 | 63 mg/24 h |
| test 2 | 79 mg/24 h |
| test 3 | 72 mg/24 h |

D/ PCL, Molecular Weight=37,000 g/mol, and PCL, Molecular Weight=10,000 g/mol, with Different Concentrations of Lidocaine

The tests complementing those described in paragraph C/ relate to the following mixtures:
test 1: 5 g of PCL (molecular weight 37,000 g/mol)+2 g of PCL (molecular weight 10,000 g/mol)+3 g of lidocaine
test 2: 5 g of PCL (molecular weight 37,000 g/mol)+3.5 g of PCL (molecular weight 10,000 g/mol)+1.5 g of lidocaine

The results are collated in Table 8.

TABLE 8

| | |
|---|---|
| test 1 | 300 mg/24 h |
| test 2 | 1000 mg/24 h |

### EXAMPLE 5

### In Vitro Kinetics for the Release of Bupivacaine from an Implant Based on PLA₅₀GA₅₀

Complementary tests on the release of bupivacaine were performed on a batch of four identical implants of parallelepipedal shape with a square base, having dimensions of 70×5×5 mm and a weight of 2.7 g and containing 50% of said local anesthetic. These tests were performed by immersing the implant in 500 cm³ of PBS (pH=7.4) at a temperature of 37° C., this time in the absence of magnetic stirring.

Magnetic stirring was only carried out at the time of the determinations in order to homogenize the liquid medium before an aliquot thereof was taken for analysis.

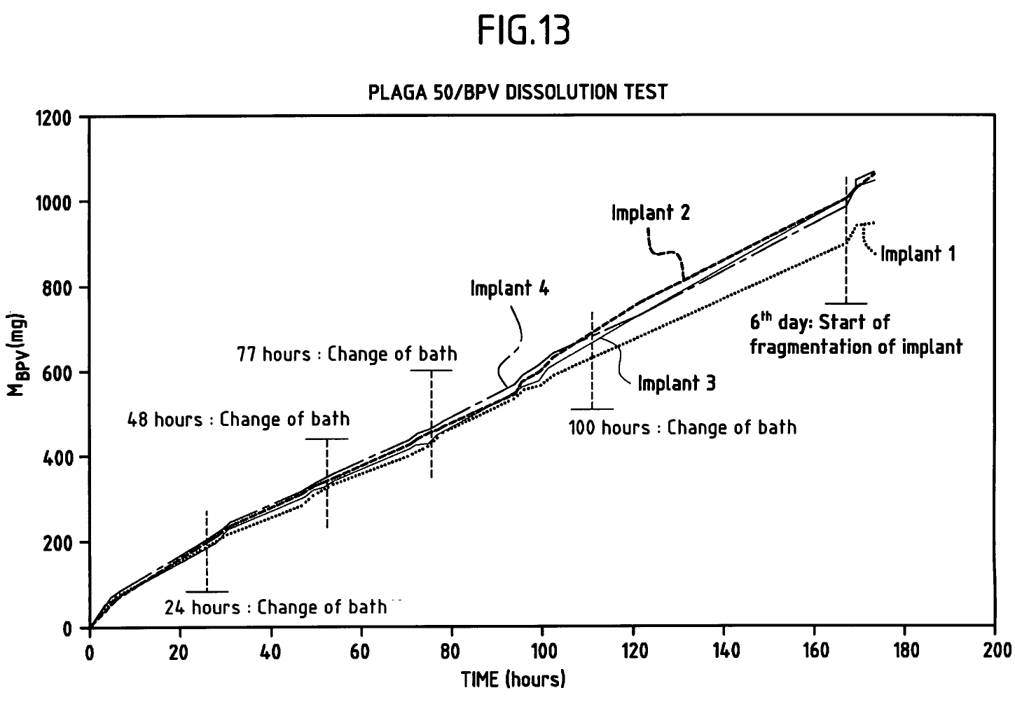

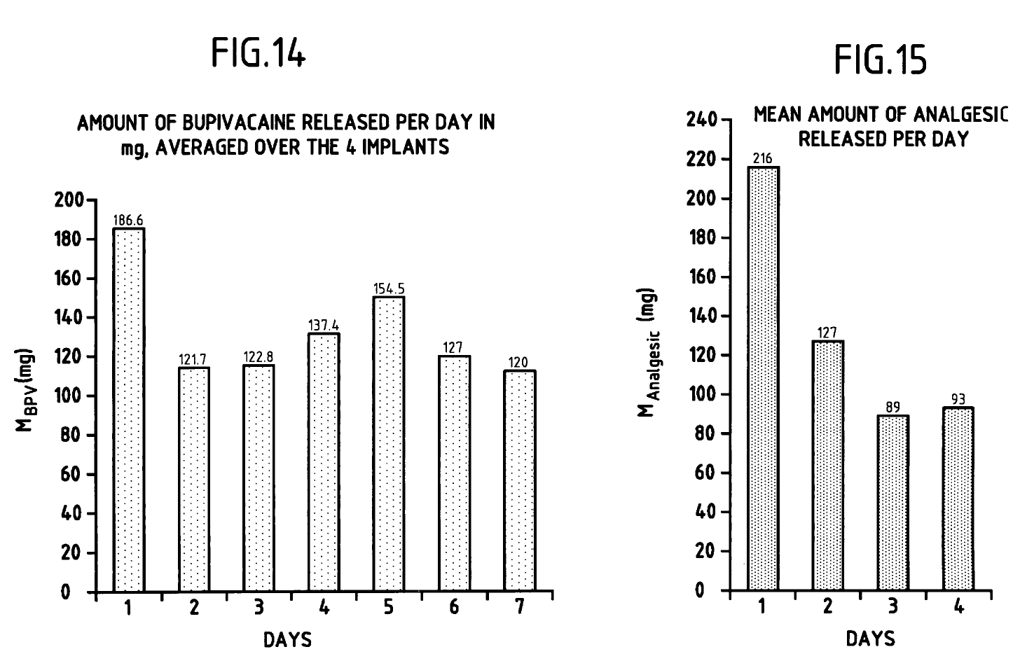

The release curve for a 6-day period is shown in FIG. 13 and is complemented by a graph showing the amount released per 24-hour period (cf. FIG. 14).

As expected, the release is greater on the first day and remains substantially constant in value on the other days of the test.

It was additionally found that the structure of the implants had been completely degraded after an immersion period of 6 days.

### EXAMPLE 6

### In Vitro Kinetics for the Release of a Mixture of Local Anesthetics from an Implant Based on PLA₅₀GA₅₀

The same experiment as that described in Example 5 was conducted on an implant containing the following concentrations of local anesthetics:
5% of lidocaine,
45% of bupivacaine,
50% of PLA₅₀GA₅₀.

The graph showing the amount released per 24-hour period is given in FIG. 15. It is noted that the release kinetics are less consistent than those observed in Example 5.

### EXAMPLE 7

### In Vitro Kinetics for the Release of Local Anesthetics from an Implant of “Sandwich” Structure Based on PLA₅₀GA₅₀

The transfer molding technique was used to produce an implant of “sandwich” structure identical to that shown in FIG. 2. The peripheral structure (the envelope) is formed of two 1.5 g ribbons whose support material is a 95/5 (by weight) mixture of PLA₅₀GA₅₀ and lactic acid and which contain lidocaine in a relative proportion of 25% by weight. The internal structure (the core) is made of the same support material but contains bupivacaine in a relative proportion of 50% by weight.

For each structure of parallelepipedal shape (length 7 cm, width 1 cm, thickness 1 mm), the time in the transfer chamber, heated to 85° C., is 15 min. The molding pressure on a Teflon-coated plate is 65 bar. Table 9 shows the data obtained.

TABLE 9

| mg/24 h | mg/48 h | mg/72 h |
|---|---|---|
| 550 | 490 | 510 |

Virtually linear release kinetics are observed.

### EXAMPLE 8

### In Vitro Kinetics for the Structural Degradation of an Implant Consisting of PLA₅₀GA₅₀/Lactic Acid/Bupivacaine

Finally, Table 10 shows the kinetics of structural degradation, in PBS (pH=7.4) heated to 37° C., of an implant of PLA₅₀GA₅₀ copolymer/lactic acid/bupivacaine (BPV) produced by the compression-transfer molding techniques. The amount of bupivacaine was kept constant at 1.5 g for an implant with a total weight of 3 g.

TABLE 10

| | 2 days | 4 days | 15 days |
|---|---|---|---|
| 85/15/BPV | material acquiring | material reducing | total solubilization |
| | a soft consistency | in volume | |
| 90/10/BPV | material acquiring | material reducing | ″ |
| | a soft consistency | in volume | |
| 95/5/BPV | material acquiring | material reducing | solubilization after |
| | a soft consistency | in volume | 10 days of |
| | | | immersion |

The mechanisms associated with these structural degradation kinetics were elucidated by using scanning electron microscopy to observe sections of ribbons produced by cryofracture.

The microscope used is a field emission scanning electron microscope (JEOL-6400F) equipped with a stage for preparation of the sample by cryofracture (OXFORD CT 1500 HF). The operating protocol is as follows:
In a freezing chamber independent of the microscope, the sample is immersed in liquid nitrogen and placed under a primary vacuum (10⁻³ torr),
   this sample is then transferred to a chamber where a secondary vacuum (10⁻⁶ torr) prevails,
   it is fractured with a blunt instrument,
   any ice found on the sample is sublimed,
   gold (100 Å) is deposited on the fractured sample,
   and finally, the sample is transferred to the microscope stage without exposure to the air.

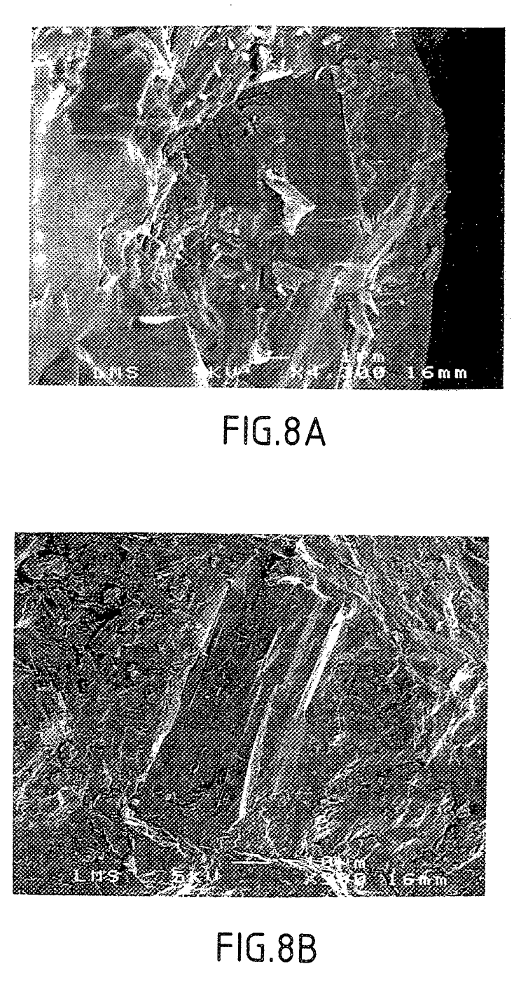

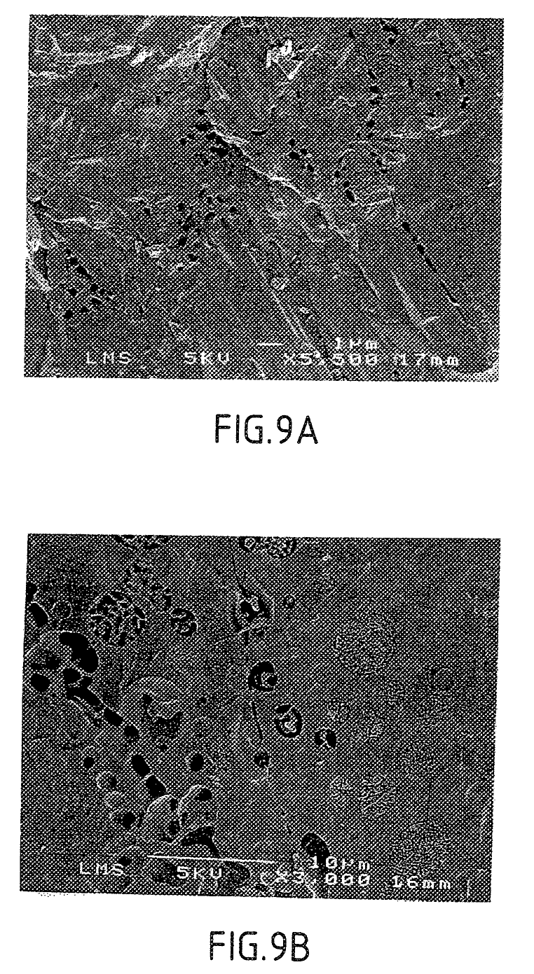

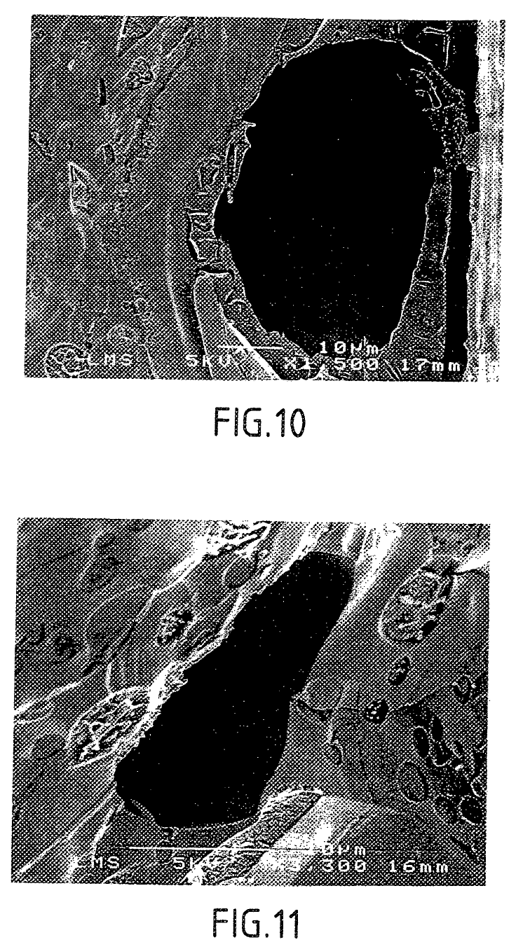

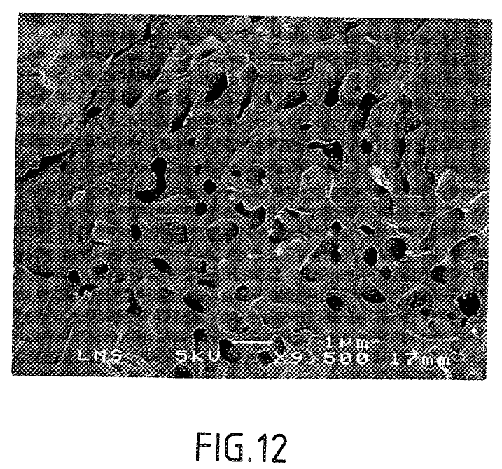

The following experimental data were thus obtained:
Firstly, a check was carried out to ensure that the density of the solid active substance (bupivacaine in the illustrated example) was homogeneous on the surface and in the core of the sample, and that the interfaces between the active substance and the polymer support appeared perfectly coherent (FIGS. 8A and 8B),
after an immersion time of a few hours (2 to 5 hours) in PBS (pH=7.4, T=37° C.), the presence of local microporosities that can be associated with the total dissolution of the active substance located on the surface of the implant is visualized (FIGS. 9A and 9B),
after an immersion time of 9 hours, a total dissolution of the active substance located on the surface of the implant is recorded (FIG. 10),
after an immersion time of 12 hours, dissolution of the active substance continues and gradually reaches the core of the material (FIG. 11),
and, concomitantly, the number of microporosities and nanoporosities in the polymer increases (FIG. 12).

Thus it seems that it is the local structural degradation of the polymer by the formation of open porosities which favors the solubilization of the active substance and hence its release over time in dissolved form.

### BIBLIOGRAPHY

1. Nehra et al. Pain relief after inguinal hernia repair: a randomized double-blind study. Br. J. Surg. 1995, 82, 1275-7.

2. Ding et al. Post-herniorraphy pain in outpatients after pre-incision ilioinguinal-hypogastric nerve block during monitored anaesthesia care. Can. J. Anaesth. 1995, 42, 12-15.

3. Reid et al. Day-case herniotomy in children. A comparison of ilio-inguinal nerve block and wound infiltration for postoperative analgesia. Anaesthesia 1987, 42, 658-61.

4. Lafferty et al. A comparison of postoperative pain relief techniques in orchidopexy. Ann. R. Coll. Surg. Engl. 1990, 72, 7-8.

5. Wright. Controlled trial of wound infiltration with Bupivacaine for postoperative pain relief after appendicectomy in children. Br. J. Surg. 1993, 80, 110-11.

6. Thomas et al. The direct perfusion of surgical wounds with local anaesthetic solution: an approach to postoperative pain? Ann. R. Coll. Surg. Engl. 1983, 65, 226-9.

7. Levack et al. Abdominal wound perfusion for the relief of postoperative pain. Br. J. Anaesth. 1986, 58, 615-19.

8. Oakley et coil. Randomized placebo-controlled trial of local anaesthetic infusion in day-case inguinal hernia repair. Br. J. Surg. 1998, 85, 797-9.

9. Griffith et coll. Prospective randomized study of a new method of providing postoperative pain relief following femoropopliteal bypass. Br. J. Surg. 1996, 83, 1735-8.

10. Fisher et Meller. Continuous postoperative regional analgesia by nerve sheath block for amputation surgery. A pilot study. Anaest. Analg. 1991, 72, 300-3.

1-19. (canceled)

20. Flexible implantable structure for the sustained and controlled release of an active principle, consisting of a bioabsorbable support and an active principle intimately associated with said support, which exhibits a cohesion between the active principle and the bioabsorbable support that is induced by the wettability of one of the components of the structure, and in which the bioabsorbable support is formed of a mixture of an amorphous lactic acid/glycolic acid copolymer having a weight ratio between the lactic acid and glycolic acid units ranging from about 80/20 to 20/80, and about 0.5 to 20% by weight, based on the weight of the support, of a biocompatible plasticizer selected from lactic acid, a lactic acid oligomer and a mixture of these compounds, said mixture of copolymer and plasticizer having a Tg below or equal to 15° C.

21. Implantable structure according to claim 20 in which the active principle is selected from local anesthetics, morphine or non-morphine analgesics, healing factors, anti-inflammatories, antibiotics, antifungals, corticoids, hormones, antimitotics, growth factors and a mixture of these active principles.

22. Implantable structure according to claim 21 in which the active principle is a local anesthetic.

23. Implantable structure according to claim 20 which is in the form of a yarn, film, hank, ribbon of parallelepipedal shape with a square or rectangular base, sliver, woven or non-woven fabric, plate, catheter, tablet, sheet or suture thread.

24. Implantable structure according to claim 20 which is in the form of a sandwich structure.

25. Implantable structure according to claim 20 in which the weight ratio between the lactic acid and glycolic acid units preferably range from about 70/30 to 30/70.

26. Implantable structure according to claim 25 in which the weight ratio between the lactic acid and glycolic acid units are about 50/50.

27. Implantable structure according to claim 20 in which the biocompatible plasticizer is 5 to 15% by weight, based on the weight of the support.

28. Process for the manufacture of a flexible implantable structure for the sustained and controlled release of an active principle, consisting of a homogeneous composite structure with coherent interfaces, in which the bioabsorbable support is formed of a mixture of an amorphous lactic acid/glycolic acid copolymer having a weight ratio between the lactic acid and glycolic acid units ranging from about 80/20 to 20/80, and about 0.5 to 20% by weight, based on the weight of the support, of a biocompatible plasticizer selected from lactic acid, a lactic acid oligomer and a mixture of these compounds, said mixture of copolymer and plasticizer having a Tg below or equal to 15° C., said process comprising the following steps:
a) mixing of the component products of the structure,
b) passage, with or without applied pressure, through a transfer chamber, either b1) at a temperature between the melting point of the active principle and the glass transition temperature or melting point of the copolymer, or b2) at a temperature that is above both the melting point of the active principle and the glass transition temperature of the copolymer, and
c) shaping of the implantable structure under pressure from this intermediate state.

29. Process according to claim 28 which also comprises d) a heat treatment step.

30. Process according to claim 28 which is a process of compression-transfer molding, injection-transfer molding, or extrusion or spinning with a preliminary transfer step.

31. Process according to claim 28 in which the mixture of products obtained in step a) is ground to give a particle size ranging from about 5 to 150 μm, preferably from about 10 to 50 μm.

32. Process according to claim 28 in which the active principle is selected from local anesthetics, morphine or non-morphine analgesics, healing factors, anti-inflammatories, antibiotics, antifungals, corticoids, hormones, antimitotics, growth factors and a mixture of these active principles.

33. Process according to claim 32 in which the active principle is a local anesthetic.

34. Process according to claim 28 in which the weight ratio between the lactic acid and glycolic acid units preferably range from about 70/30 to 30/70.

35. Process according to claim 34 in which the weight ratio between the lactic acid and glycolic acid units are about 50/50.

36. Process according to claim 28 in which the biocompatible plasticizer is 5 to 15% by weight, based on the weight of the support.
